(19) United States
(12) Patent Application Publication
(10) Pub. No.: US 2020/0154999 A1
(43) Pub. Date: May 21, 2020

(54) MULTIMODAL INTERFEROMETRIC TEAR FILM MEASUREMENT

(71) Applicant: THE UAB RESEARCH FOUNDATION, Birmingham, AL (US)

(72) Inventors: Jason J. NICHOLS, Mountain Brook, AL (US); Yuqiang BAI, Vestavia, AL (US)

(21) Appl. No.: 16/626,673

(22) PCT Filed: Jun. 26, 2018
(86) PCT No.: PCT/US2018/039598

§ 371 Date: Dec. 26, 2019

Related U.S. Application Data
(60) Provisional application No. 62/525,407, filed on Jun. 27, 2017.

(51) Int. Cl. A61B 3/18; A61B 3/10

(52) U.S. Cl. CPC A61B 3/18; A61B 3/1005; A61B 3/101; A61B 3/102

(57) ABSTRACT

A multimodal interferometric tear film measurement system can include an optical coherence tomography (OCT) system and a thickness dependent fringe (TDF) system. The multimodal interferometric tear film measurement system (multimodal interferometry system) can include a hot mirror dual focusing system that allows the OCT system's light signals and the TDF system's light signals to be individually focused while both are simultaneously directed towards the eye. The multimodal interferometry system can provide OCT and TDF measurements simultaneously. The OCT system can provide measurements of the thickness of the entire tear film, while the TDF system can provide measurements of the thickness of the lipid layer of the tear film. The multimodal interferometry system can provide in vivo measurements of both lipid layer and overall tear film.

### CROSS REFERENCE TO RELATED APPLICATIONS

The present application claims the benefit of U.S. Provisional Patent Application No. 62/525,407 filed Jun. 27, 2017 and entitled “MULTIMODAL INTERFEROMETRIC TEAR FILM MEASUREMENT,” which is hereby incorporated by reference in its entirety.

### TECHNICAL FIELD

The present disclosure relates to medical diagnostic equipment generally and more specifically to interferometric measurement of ocular tear films.

### BACKGROUND

The eye is generally protected by a tear film made up of multiple layers and components. The outermost layer is a lipid layer, which primarily seals the tear film and prevents evaporation of the tear film. The lipid layer can be approximately 0.1-0.2 micrometers in thickness. An aqueous layer lies beneath the lipid layer, which primarily lubricates the eye, prevents infection, and facilitates removal of debris. The aqueous layer can be approximately 3-4 micrometers in thickness. An innermost mucin layer acts as a wetting agent to facilitate distribution of the aqueous layer. The mucin layer can be approximately 1 micrometer in thickness. The tear film itself rests upon the cornea. With each blink, the tear film changes, as new tears enter the film and the eyelid slides against the surface of the existing tear film.

Irregularities in the tear film can lead to various undesirable symptoms and conditions. For example, certain problems with tear production or tear quality can lead to an insufficient tear film eliciting symptoms of dry eyes, such as irritation and burning, or even potential corneal damage and vision impairment.

Measuring the tear film can be difficult, especially due to the ever-changing and dynamic nature of the tear film and exposure to the atmosphere of this mucous membrane. During the period of only a few seconds or less, a tear film can change due to evaporation, blinking, or other actions. Existing technologies have largely proven unable to simultaneously provide accurate measurements of full tear film thickness and lipid layer thickness. Further, existing technologies are generally only able to take measurements of small areas of the tear film, such as areas on the order of a few micrometers in diameter.

Accurate measurement of characteristics of the tear film can provide useful insight necessary to understand how the tear film functions, as well as any related systems. Additionally, accurate measurement of the tear film can improve the ability to diagnose patients, the ability to select the most desirable treatment plan, and the ability to track the efficacy of treatment plans, among other uses.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a multimodal interferometric system, comprising a first interferometric imaging system having a port for outputting a first light wave and receiving a first reflected light wave; a second interferometric imaging system having a port for outputting a second light wave and receiving a second reflected light wave, wherein the first light wave and the second light wave have different wavelengths; and a dual focusing system having: a first port optically coupled to the port of the first interferometric imaging system; a second port optically coupled to the port of the second interferometric imaging system; and a dichoric mirror optically coupled to the first port through a first focusing lens and the second port through a second focusing lens, wherein the dichoric mirror is selected to reflect the first light wave and the first reflected light wave while allowing light from the second light wave and the second reflected light wave to pass therethrough, wherein the dichoric mirror is positioned to combine the first light wave and the second light wave into a combined light wave and direct the combined light wave through an objective lens, wherein the first light wave passing through the first focusing lens and the objective lens is focused at a first focal plane, wherein the second light wave passing through the second focusing lens and the objective lens is focused at a second focal plane, and wherein the first focal plane and second focal plane are spaced apart from one another.

In some cases, the first interferometric imaging system is an optical coherence tomography system including a low-coherence light source for generating the first light wave and a beam coupler for combining the first reflected light wave and a reference light wave. In some cases, the second interferometric imaging system is a thickness dependent fringe system including a light source for generating the second light wave and a beam splitter for directing the second light wave from the light source to the port of the second interferometric imaging system, wherein the beam splitter is positioned to permit the second reflected light wave to pass therethrough and into a sensor. In some cases, the first focal plane is positionable at a surface of a tear film of an eye, and wherein the second focal plane is positionable within the eye at a distance beyond the surface of the tear film of the eye. In some cases, a distance between the first focal plane and the second focal plane is between one millimeter and fifteen millimeters. In some cases, the first light wave has a first wavelength that is longer than a second wavelength of the second light wave, and wherein the dichoric mirror is a hot mirror that reflects light at the first wavelength and passes through light at the second wavelength. In some cases, the system also includes a processor coupled to the first interferometric imaging system and the second interferometric imaging system to synchronously initiate interferometric measurements from both the first interferometric imaging system and the second interferometric imaging system.

Embodiments of the present disclosure include a multimodal objective for simultaneous, multimodal interferometry, the multimodal objective comprising a first port optically couplable to a first interferometric imaging system for receiving a first light wave and transmitting a first reflected light wave; a second port optically couplable to a second interferometric imaging system for receiving a second light wave and transmitting a second reflected light wave, wherein the first light wave and the second light wave have different wavelengths; and a dichoric mirror optically coupled to the first port through a first focusing lens and the second port through a second focusing lens, wherein the dichoric mirror is selected to reflect the first light wave and the first reflected light wave while allowing light from the second light wave and the second reflected light wave to pass therethrough, wherein the dichoric mirror is positioned to combine the first light wave and the second light wave into a combined light wave and direct the combined light wave through an objective lens, wherein the first light wave passing through the first focusing lens and the objective lens is focused at a first focal plane, wherein the second light wave passing through the second focusing lens and the objective lens is focused at a second focal plane, and wherein the first focal plane and second focal plane are spaced apart from one another.

In some cases, the first focal plane is positionable at a surface of a tear film of an eye, and wherein the second focal plane is positionable within the eye at a distance beyond the surface of the tear film of the eye. In some cases, a distance between the first focal plane and the second focal plane is between one millimeter and fifteen millimeters. In some cases, the first light wave has a first wavelength that is longer than a second wavelength of the second light wave, and the dichoric mirror is a hot mirror that reflects light at the first wavelength and passes through light at the second wavelength. In some cases, the objective lens is an achromatic lens. In some cases, the first focusing lens and the second focusing lens are individually adjustable to respectively adjust the first focal plane and the second focal plane.

Embodiments of the present disclosure include a method for performing simultaneous, multimodal interferometry, the method comprising receiving a first light wave at a first port and a second light wave at a second port, wherein the first light wave is associated with a first interferometric technique and the second light wave is associated with a second interferometric technique; directing the first light wave to a dichoric mirror through a first focusing lens; directing the second light wave to the dichoric mirror through a second focusing lens; combining the first light wave and the second light wave using the dichoric mirror and directing the combined light to an objective lens, wherein the combined light includes a first component from the first light wave and a second component from the second light wave; outputting the combined light through the objective lens, wherein the first focusing lens and the objective lens focus the first component of the combined light onto a first focal plane, wherein the second focusing lens and the objective lens focus the second component of the combined light onto a second focal plane, and wherein the first focal plane and the second focal plane are spaced apart from one another; receiving combined reflected light at the objective lens and directing the combined reflected light to the dichoric mirror, wherein the combined reflected light is the combined light reflected off a subject; splitting the combined reflected light into a first reflected light wave and a second reflected light wave using the dichoric mirror, wherein the first reflected light is directed through the first focusing lens, and wherein the second reflected light is directed through the second focusing lens; and outputting the first reflected light at the first port and the second reflected light at the second port, wherein outputting of the first reflected light results in measurement data according to the first interferometric technique, and wherein outputting of the second reflected light results in measurement data according to the second interferometric technique.

In some cases, receiving the first light wave at the first port and the second light wave at the second port occurs simultaneously. In some cases, the method further comprises generating the first light wave at a low-coherence light source of an optical coherence tomography system; generating a reference light wave using the first light wave; combining the first reflected light wave and the reference light wave into a first combined measurement wave; and measuring interference patterns in the first combined measurement wave. In some cases, the method further comprises generating the second light wave at a light source of a thickness dependent fringe interferometry system; reflecting the second light wave off a beam splitter and into the second port; and passing the second reflected light wave through the beam splitter and into a sensor. In some cases, the method further comprises maneuvering the first focusing lens, the second focusing lens, and the objective lens to position the first focal plane at a surface of a tear film of an eye and the second focal plane within the eye at a distance beyond the surface of the tear film of the eye. In some cases, a distance between the first focal plane and the second focal plane is between one millimeter and fifteen millimeters. In some cases, the method further comprises receiving a trigger signal, wherein receiving the trigger signal initiates a simultaneous measurement process and results in simultaneously receiving the first light wave at the first port and the second light wave at the second port; and simultaneously transmitting the measurement data associated with the first interferometric technique and the measurement data associated with the second interferometric technique.

### BRIEF DESCRIPTION OF THE DRAWINGS

The specification makes reference to the following appended figures, in which use of like reference numerals in different figures is intended to illustrate like or analogous components.

FIG. 1 is a schematic diagram depicting a multimodal interferometric tear film measurement system according to certain aspects of the present disclosure.

FIG. 2 is a schematic diagram depicting a multimodal interferometric tear film measurement system having an optical coherence tomography system and a thickness dependent fringe system according to certain aspects of the present disclosure.

FIG. 3 is a schematic diagram depicting a hot mirror dual focusing system according to certain aspects of the present disclosure.

FIG. 4 is an image of a tear film surface taken by a thickness dependent fringe system of a multimodal interferometric tear film measurement system according to certain aspects of the present disclosure.

FIG. 5 is a graphic representation of a an image of a tear film surface taken by a thickness dependent fringe system of a multimodal interferometric tear film measurement system according to certain aspects of the present disclosure.

FIG. 6 is a cross-sectional image of a tear film as taken by an optical coherence tomography system of a multimodal interferometric tear film measurement system according to certain aspects of the present disclosure.

FIG. 7 is a graphical representation of a cross-sectional image of a tear film as taken by an optical coherence tomography system of a multimodal interferometric tear film measurement system according to certain aspects of the present disclosure.

### DETAILED DESCRIPTION

Certain aspects and features of the present disclosure relate to a multimodal interferometric tear film measurement system including an optical coherence tomography (OCT) system and a thickness dependent fringe (TDF) system. As used herein, the term multimodal can refer to the ability of a single system to operate both OCT and TDF interferometry from a single objective, such as simultaneously or sequentially. The multimodal interferometric tear film measurement system (multimodal interferometry system) can include a hot mirror dual focusing system that allows the OCT system's light signals and the TDF system's light signals to be individually focused while both are simultaneously directed towards the eye. The multimodal interferometry system can provide OCT and TDF measurements sequentially, simultaneously, or nearly-simultaneously (e.g., within 50 ms, 100 ms, 200 ms, 500 ms, 750 ms, 1 s, 1.5 s, 2 s, or 5 s). The OCT system can provide measurements of the thickness of the entire tear film and its rates of thinning, while the TDF system can provide measurements of the thickness of the lipid layer of the tear film (e.g., the outermost layer of the tear film). The multimodal interferometry system can provide in vivo measurements of both lipid layer and overall tear film thickness, thinning/decay and distribution. The measured thicknesses can be used for dry eye diagnosis, evaluation of evaporative dry eye disease, and evaluation and monitoring of clinical treatments for these conditions.

Optical coherence tomography (OCT) can include ultra-high resolution optical coherence tomography. A broad bandwidth source in the near infrared region can be directed to the eye to measure the whole thickness of the tear film. In some cases, the light source for the OCT system can be a low-coherence light source, such as a superluminescent diode. The OCT system can use a 2×2 coupling fiber, a reference arm (e.g., including a reference mirror and appropriate optics), an imaging lens with a low numerical aperture (e.g., narrow cone of focus), and a spectrometer with a diffraction grating. Light from the light source can be split into a sample path and a reference path. Measurements of the sample (e.g., tear film) can be determined by detecting the interference between light reflected from the sample and light reflected from reference mirror. The OCT system can be a time-domain interferometer (e.g., using a scanning reference mirror to determine the precise position of the reflection point in the sample) or a frequency domain interferometer (e.g., using a combination of diffraction grating and line scan camera to spectrally decompose interferogram or using a sweeping laser source to capture the spectrum of the interference pattern in time). At a single location, the peaks in reflectivity profiles (e.g., A-scans) can be used to identify the cornea-tear film boundary and the air-tear film boundary. The thickness of the tear film directly obtained from OCT A-scans can be divided by the average group refractive index of the tear film (e.g., 1.339). The light path can be scanned across the sample (e.g., scanned in X and Y directions along a plane perpendicular to the direction of the light path) to image an area larger than the light beam itself. Rapid scanning can produce a measurement over a larger area, such as diameters on the scale of tens or hundreds of micrometers or several millimeters (e.g., at or greater than 50 μm, 100 μm, 500 μm, 1 mm, 2 mm, 3 mm, 4 mm, or 5 mm). The OCT system can output measurements and/or images of the thickness of the entire tear film (e.g., between the cornea and the outer surface of the lipid layer).

Thickness dependent fringe (TDF) interferometry can involve directing a broad light source towards a sample (e.g., the surface of the tear film) and measuring interference fringes caused by the incident light interfering with the reflected light. The light can be in the visible region (e.g., light having wavelengths between approximately 390 nm to approximately 700 nm), although ranges can be used. Any suitable light source can be used, such as a tungsten-halide source. Light from the light source can be reflected off a glass plate beam splitter and pass through an objective (e.g., objective lens) having a high numerical aperture (e.g., wide cone of focus) to focus the light at the apex of the cornea. Reflected light can pass through the objective and the glass plate beam splitter and into a receiver (e.g., camera). Wavelength-dependent fringes can be analyzed to determine thickness of the lipid layer of the tear film. In other words, the thickness of the lipid layer can be observed as differences in color received at the camera. The light can cover a wide surface area, allowing for a wide surface area to be measured at any point in time, such as diameters on the scale of tens or hundreds of micrometers or several millimeters (e.g., an area at or greater than 50 μm, 100 μm, 500 μm, 1 mm, 2 mm, 3 mm, 4 mm, or 5 mm). The TDF system can output measurements and/or images representing the thickness of the lipid layer of the tear film (e.g., the outermost layer of the tear film).

When used alone, an OCT system can be suitable for measuring thickness of the whole tear film and cornea, however can lack sufficient resolution to provide meaningful insight into the condition of the lipid layer of the tear film. When used alone, a TDF system can be suitable for measuring thickness of the outermost lipid layer of the tear film, however can lack sufficient penetration to provide meaningful insight into the condition of the entire tear film.

The ability to take OCT and TDF measurements simultaneously provides numerous benefits. The measurements and/or images from both systems may be time-correlated to one another, allowing for the simultaneously monitoring of tear film conditions at the outmost layer (e.g., the lipid layer) and the overall tear film (e.g., thinning of the aqueous layer). Since some diagnostics are dependent on events occurring over time, the ability to view measurements over time of both the full tear film and the outermost layer of the tear film can be beneficial. For example, debris and dry spots may appear similar on a single image of the lipid layer, however debris may move with successive blinks, whereas dry spots may remain stationary. Thus, the ability to view data over time can provide for improved diagnostic ability. In previous systems, a user would have to switch between an OCT system and a TDF system in order to measure both the full tear film and the lipid layer. This time delay during switching decouples the OCT measurements from the TDF measurements. During the time delay, either the patient would have to keep from blinking, in which case the tear film may dry out due to evaporation and may collect debris from the surrounding environment, or the patient would blink, thus chancing the tear film through movement of the eyelid and the addition of near tears. The configuration of the multimodal interferometry system allows for enhanced information for the diagnose of dry eye disease and ocular surface conditions (e.g., contact lens wear, meibomian gland dysfunction, ocular allergy, corneal irregularities, and others), which may be due to a number of deficiencies, including lipid deficiency and aqueous deficiency. The multimodal interferometry system may have further benefits not described herein.

Additionally, certain aspects and features of the present disclosure allow for accurate, simultaneous measurement of lipid layer thickness and whole tear film thickness without the need to take multiple, consecutive images to reduce background noise. Further, certain aspects and features of the present disclosure allow for accurate measurement of the lipid layer simultaneously with the whole film tear without relying on statistical estimation from OCT measurements alone to estimate lipid layer thickness.

In some cases, the multimodal interferometry system can combine OCT and TDF systems using a dichroic mirror and two commercial objectives (e.g., objective lenses). However, the insertion of an external dichroic mirror can sacrifice the working distance of the objectives. A significant distance between the objective and the subjects may be desirable in many applications. Also the combination of two objectives with one external mirror is difficult to produce.

The present multimodal interferometry system combines OCT and TDF techniques into a multimodal instrumentation that provides benefits far beyond the individual OCT and TDF systems when used alone. The multimodal interferometry system can include a specific hot mirror dual focusing system capable of providing simultaneous, optimal focusing for each of the OCT and TDF systems without detracting from the overall functionality of each system. The hot mirror dual focusing system is capable of combining together the two detecting beams of the OCT and TDF systems while maintaining their individual requirements in wavelength range and depth of focus, allowing for the measurements to be taken simultaneously. The hot mirror dual focusing system can act as an objective for both the OCT and TDF systems, taking in two inputs (e.g., one for the OCT signal and one for the TDF signal) and use an internal dichoric mirror to combine the beams together into a common output. As used herein with reference to the hot mirror dual focusing system, the terms “input” and “output” are used for convenience and may refer to the direction of the outgoing light exiting the OCT and TDF systems, however it is understood that reflected light from the eye will enter the hot mirror dual focusing system through the “output” and exit through the appropriate “inputs” to be directed back into the OCT and TDF systems for analysis.

As used herein, the terms OCT system and TDF system can include the equipment necessary to perform OCT techniques and TDF techniques, respectively, with or without objectives (e.g., objective lenses and other optics) necessary to direct the measurement beams to an eye. The hot mirror dual focusing system can function as the objectives for both the OCT system and TDF system to focus and direct the measurement beams for both the OCT system and the TDF system to the eye.

In some cases, the OCT system or TDF system can be customary OCT or TDF measurement systems that have been modified to operate with the hot mirror dual focusing system. In some cases, purpose built OCT and/or TDF systems can be used in the multimodal interferometry system.

OCT and TDF techniques each have different illumination methods and focusing requirements. A TDF system can illuminate the ocular surface with parallel light rather than a broad illumination cone. Such a configuration may slightly reduce the potential resolution, but can enable more tolerance for defocusing, given that the front surface of eyes is curved. For example, in some cases, the objective of TDF can have a working distance of at or approximately 4 cm, while the image plane of tear film is placed at a distance of approximately 3 cm to the objective (e.g., the distance between tear film and the focus of the objective is at or approximately 1 cm). On the contrary, an OCT system can image the tear film exactly at or as near as possible to the focus of objective. Therefore, merely combining an OCT system and a TDF system with a common objective would require compromising the focusing requirements of one or both of the systems.

The hot mirror dual focusing system disclosed herein can accommodate for the different illumination methods and focusing requirements of the OCT and TDF techniques. The hot mirror dual focusing system provides separate focusing lenses for the OCT and TDF systems at respective input ports, allowing the beam of each system to converge at different extents respectively. Then, after combining the differently-converging beams using a dichroic mirror, the combined beam is passed through a multielement lens to focus the combined beam on the eye such that the illumination and focusing requirements of the OCT and TDF systems are both maintained. As necessary, adjustments can be made in the individual focusing lenses to control the focal planes of the respective OCT and TDF systems. For example, adjustment of a first focusing lens with respect to the common lens can focus the OCT signal at the tear film, enabling point-scanning algorithm, while adjustment of a second focusing lens with respect to the common lens can focus the TDF signal into the cornea (e.g., around 1 cm behind the tear film layer). The common lens can be a multi-element lens to minimize optical aberration, such as chromatic, spherical aberration, coma, astigmatism, and field curvature.

The combined measurement system can include and/or be controlled by one or more processors, such as one or more general-purpose processors and/or one or more special-purpose processors (e.g., digital signal processing chips, graphics acceleration processors, and/or the like). Software can be used to control the functionality of the combined measurement system. Software can be stored on memory, such as on a non-transitory machine-readable storage medium, on processor-readable memory, and/or on a computer-readable memory that stores the one or more computer-program products configured to cause the processor to perform the various functions described herein. The software can control the synchronization of the OCT and TDF systems. In some cases, the OCT and TDF systems can operate simultaneously or near-simultaneously (e.g., within 50 ms, 100 ms, 200 ms, 500 ms, 750 ms, 1 s, 1.5 s, 2 s, or 5 s). In some cases, the multimodal interferometry system can operate one of the OCT and TDF systems at a time, allowing for easy switching between the two modes of operation on demand or automatically, without requiring the patient or the equipment to be moved. The software can allow for automated measurements to be taken, simultaneously or sequentially, based on pre-determined instructions or active feedback (e.g., based on blinking detected by an operatively coupled sensor, such as a sensor of the OCT or TDF systems or a separate sensor).

Certain aspects and features of the present disclosure enable the simultaneous measurement of the thicknesses of the lipid and aqueous layers, which can be used in the diagnosis of a patient's tear film, including but not limited to lipid layer and aqueous layer deficiencies. The measured characteristics may be a cause, contributing factor, or symptom associated with a patient experiencing dry eye syndrome or other affliction.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

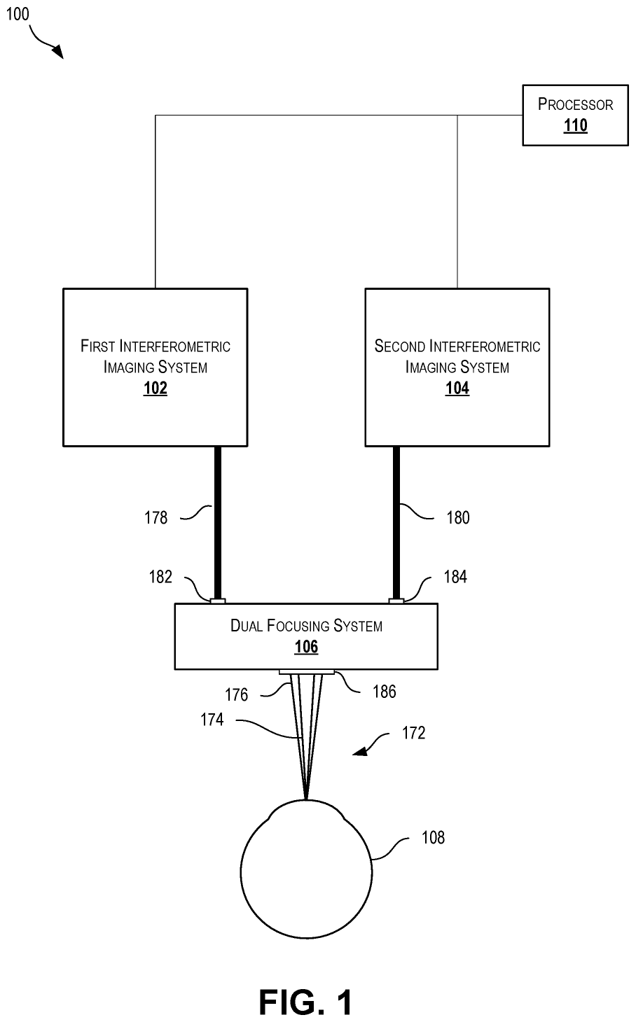

FIG. 1 is a schematic diagram depicting a multimodal interferometric tear film measurement system 100 (multimodal interferometry system) according to certain aspects of the present disclosure. The multimodal interferometry system 100 can include a first interferometric imaging system 102 and a second interferometric imaging system 104 optically coupled to a dual focusing system 106 through respective optical couplings 178, 180. As used herein, the term “interferometric imaging system” can include a system that generates visual images (e.g., a visual depiction of tear film thickness) and/or a system that generates data measurements without accompanying visual images (e.g., numerical data of tear film thickness). Each of the first and second interferometric imaging systems 102, 104 can generate measured data (e.g., numeric data or visual data) by analyzing interference associated with light transmitted through and light received through the respective optical couplings 178, 180

The dual focusing system 106 is capable of combining received light from both the first interferometric imaging system 102 and the second interferometric imaging system 104 into a combined light beam 172. The dual focusing system 106 can receive the light from the first and second interferometric imaging systems 102, 104 through first and second ports 182, 184, respectively. The combined light beam 172 can exit the dual focusing system 106 through a common objective lens 186. The common objective lens 186 can be an achromatic lens. The common objective lens 186 can be a multiple element lens. The combined light beam 172 can include a first component 174 associated with the first interferometric imaging system 102 and a second component 176 associated with the second interferometric imaging system 104. The combined light beam 172 can reflect off a sample to be measured, such as a tear film of an eye 108. The dual focusing system 106 can allow the first component 174 to be focused at a different focal plane than the second component 176, while both the first component 174 and the second component 176 share a common objective lens 186. The dual focusing system 106 can receive reflected light that has been reflected off the sample (e.g., eye 108) from the combined light beam 172 and split that combined reflected light into distinct reflected light waves associated with respective ones of the first interferometric imaging system 102 and the second interferometric imaging system 104.

The multimodal interferometry system 100 can allow a first and second interferometric imaging systems 102, 104 to operate simultaneously, near-simultaneously, or sequentially. The multimodal interferometry system 100 can allow a first and second interferometric imaging systems 102, 104 to operate out of the same objective lens 186. The multimodal interferometry system 100 can allow a first and second interferometric imaging systems 102, 104 to operate out of the same objective lens 186, despite having differing illumination methodologies and/or focusing requirements.

In some cases, the first interferometric imaging system 102 can be an optical coherence tomography system. The first component 174 of the combined light beam 172 can be associated with the optical coherence tomography system and can include low-coherence light focused at a surface of the tear film of the eye 108. In some cases, the second interferometric imaging system 104 can be a thickness dependent fringe system. The second component 176 of the combined light beam 172 can be associated with the thickness dependent fringe system and can include light focused at a focal plane within the eye 108 and beyond the surface of the tear film of the eye 108, such as at distance of approximately 1 mm, 2 mm, or 3 mm beyond the surface of the tear film of the eye 108. In some cases, the focal plane of the second component 176 can be located beyond the surface of the tear film of the eye 108 by a distance of at or approximately 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, 11.5 mm, 12 mm, 12.5 mm, 13 mm, 13.5 mm, 14 mm, 14.5 mm, or 15 mm. In some cases, the focal plane of the second component 176 can be located beyond the surface of the tear film of the eye 108 by a distance between 0.1 mm and 15 mm, 0.5 mm and 3.5 mm, 1 mm and 3 mm, or 1 mm and 2 mm. Other distances may be used as appropriate.

In some cases, the multimodal interferometry system 100 can optionally include a processor 110 coupled to the first interferometric imaging system 102 and the second interferometric imaging system 104. The processor 110 can control the initiation of measurements and/or can receive measured data from the first and second interferometric imaging systems 102, 104. In some cases, the processor 110 can provide one or more trigger signals to initiate measurements by the first and second interferometric imaging systems 102, 104. A trigger signal can cause one or both of the interferometric imaging systems 102, 104 to begin transmitting and collecting light for the purpose of generating an interferometric measurement. In some cases, one or more trigger signals can cause the first and second interferometric imaging systems 102, 104 to operate simultaneously or near-simultaneously. In some cases, one or more trigger signals can cause the first and second interferometric imaging systems 102, 104 to operate sequentially. In some cases, the processor 110 can receive measured data from the first and second interferometric imaging system 102, 104 and time-stamp the data. In some cases, the processor 110 can receive measured data from the first and second interferometric imaging system 102, 104 and time-synchronize the data. In such cases, the processor 110 can facilitate reading or playing back the measured data from both the first and second interferometric imaging system 102, 104 synchronously. For example, a user viewing live or saved data associated with the first and second interferometric imaging systems 102, 104 can be presented with synchronized images of the entire tear film thickness (e.g., as collected via the OCT system) and the lipid layer thickness (e.g., as collected via the TDF system). The processor 110 can be operably coupled to a storage device to store the measured data, program instructions, or any other suitable information.

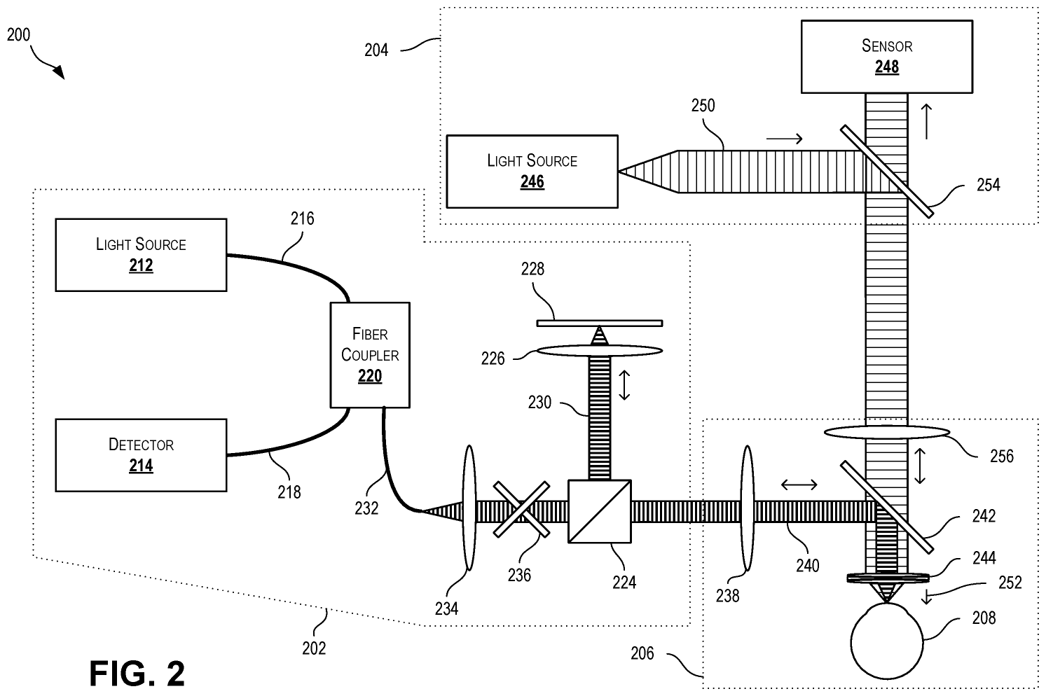

FIG. 2 is a schematic diagram depicting a multimodal interferometric tear film measurement system 200 having an optical coherence tomography system 202 and a thickness dependent fringe system 204 according to certain aspects of the present disclosure. The optical coherence tomography system 202 and the thickness dependent fringe system 204 are optically coupled to the dual focusing system 206. The multimodal interferometry system 200 of FIG. 2 can be the multimodal interferometry system 100 of FIG. 1.

The optical coherence tomography system 202 described herein involves light being directed along a number of paths. Each of the paths described herein can include one or more optical fibers for transmitting the light from one component to another, as well as any additional suitable optical equipment, such as additional lenses and mirrors. In some cases, no optical fibers are used.

The optical coherence tomography system 202 can include a light source 212 capable of generating low-coherence light, such as a laser or a superluminescent diode, although other types of light sources can be used. The light source 212 can operate in large wavelength light, such as within the infrared region of the light spectrum (e.g., between approximately 700 nm to 1,000,000 nm in wavelength), although other wavelengths can be used. Light exiting the light source 212 can pass along source path 216 and into a fiber coupler 220.

Light from the fiber coupler 220 can pass through the sample path 232 towards a collimator 234 and through a scanning mirror 236 to a beam splitter 224. The scanning mirror 236 can allow the light to be scanned in X and Y directions to perform measurements over a larger surface area than the diameter of a single beam of light. The beam splitter 224, can split the light into a reference light 230 and an OCT sample light 240. Any suitable beam splitter can be used, such as a beam splitter cube. The reference light 230 can be directed through a reference objective lens 226 and onto a reference mirror 228. The reference mirror 228 can be mounted stationary or can be axially movable in a direction perpendicular to the reflecting surface of the reference mirror 228 (e.g., movable in a direction generally parallel to the direction of travel of the reference light 230). The reference light 230 can impact the reference mirror 228 and bounce back towards the beam splitter 224, as indicated by the two-way arrow in FIG. 2. When a frequency domain interferometer is used, the reference mirror 228 may be held stationary. When a time-domain interferometer is used, the reference mirror 228 may be moved axially as necessary.

The OCT sample light 240 from the beam splitter 224 can then be directed to the dual focusing system 206. As described herein, OCT sample light 240 entering the dual focusing system 206 can be directed to a sample 208, such as an eye, and reflected OCT sample light 240 can be directed back to the optical coherence tomography system 202, as indicated by the two-way arrow in FIG. 2. Upon returning to the beam splitter, the reflected OCT sample light 240 can combine with the reflected reference light 230 and travel back towards the fiber coupler 220 and out into the detector 214 through a detection path 218. The detector 214 can be a spectrometer or other suitable piece of equipment for measuring or detection interference patterns between the reflected light 230 and the reflected OCT sample light 240. Measurements made by the detector 214 can be transferred to a computer or other processor for analysis. The elements in FIG. 2 are not drawn to scale. The distance the light travels along the reference leg (e.g., from the beam splitter 224 to the reference mirror 228 and back to the beam splitter 224) can be the same as the distance the light travels along the sample leg (e.g., from the beam splitter 224 to the sample 208 and back to the beam splitter 224).

In some cases, the optical coherence tomography system 202 can take on different forms and can include different or additional optical elements while still directing light towards and receiving light from the dual focusing system 206.

The multimodal interferometry system 200 can also include a thickness dependent fringe system 204. In the thickness dependent fringe system 204, a light source 246 can generate a TDF sample light 250. The light source 246 can generate light across a broad spectrum of wavelengths, such as across most or as least a portion of the visible light spectrum (e.g., approximately 400 nm to 700 nm), although other ranges can be used. In some cases, the light source 246 generates white light. Any suitable light source 246 can be used, such as a tungsten-halide lamp. The TDF sample light 250 can travel towards a beam splitter 254, such as a glass plate beam splitter. The TDF sample light 250 can be reflected into the dual focusing system 206. As described herein, the TDF sample light 250 entering the dual focusing system 206 can be directed to a sample 208, such as an eye, and reflected source light 240 can directed back into the thickness dependent fringe system 204 and towards the beam splitter 254. At least a portion of the reflected source light 240 can pass through the beam splitter 254 and into a sensor 248. In some case, a lens (not shown) may be used to focus the reflected source light 240 onto the sensor 248. The sensor 248 can be any suitable sensor, such as a camera or other image sensor. The direction of travel of the sensed light is shown by the one-way and two-way arrows adjacent the TDF sample light 250 in FIG. 2.

The dual focusing system 206 can receive the OCT sample light 240 and the TDF sample light 250. The OCT sample light 240 can be directed through an OCT focusing lens 238 and into a dichoric mirror 242. The TDF sample light 250 can be directed through a TDF focusing lens 256 and into the dichoric mirror 242. The dichoric mirror 242 can be selected to reflect the OCT sample light 240 while allowing the TDF sample light 250 to pass therethrough. In some cases, the dichoric mirror 242 is a hot mirror designed to reflect infrared light and pass visible light therethrough to thus reflect infrared OCT sample light 240 and pass through white TDF sample light 250. The ports, focusing lenses 238, 256, and dichoric mirror 242 can be oriented such that the incoming TDF sample light 250 is oriented perpendicular to the OCT sample light 240. A measurement direction 252 can be defined as the direction from the objective lens 244 towards the sample 208 (e.g., eye) during measurement. The TDF sample light 250 can be oriented parallel to the measurement direction 252 and the incoming OCT sample light 240 can be oriented perpendicular to the measurement direction 252 until it has been reflected off the dichoric mirror 242, after which it can be oriented parallel to the measurement direction 252. The dichoric mirror 242 can combine the OCT sample light 240 and TDF sample light 250 into a combined light beam directed through the objective lens 244 and onto the sample 208. Reflections from the sample 208 can then pass back through the objective lens 244 and onto the dichoric mirror 242, where the OCT component of the combined light beam can be reflected back towards the optical coherence tomography system 202 through the OCT focusing lens 238 and where the TDF component of the combined light beam can pass through the dichoric mirror 242 and back into the thickness dependent fringe system 204 through the TDF focusing lens 256.

In some cases, the dichoric mirror 242 can be selected to reflect short wavelengths (e.g., visible light) and pass long wavelengths (e.g., infrared light), in which case the dual focusing system 206 can be oriented to receive the OCT sample light 240 in a direction parallel the measurement direction 252 and the TDF sample light 250 can be received in a direction perpendicular to the measurement direction 252 until it is reflected off the dichoric mirror 242 and towards the sample 208.

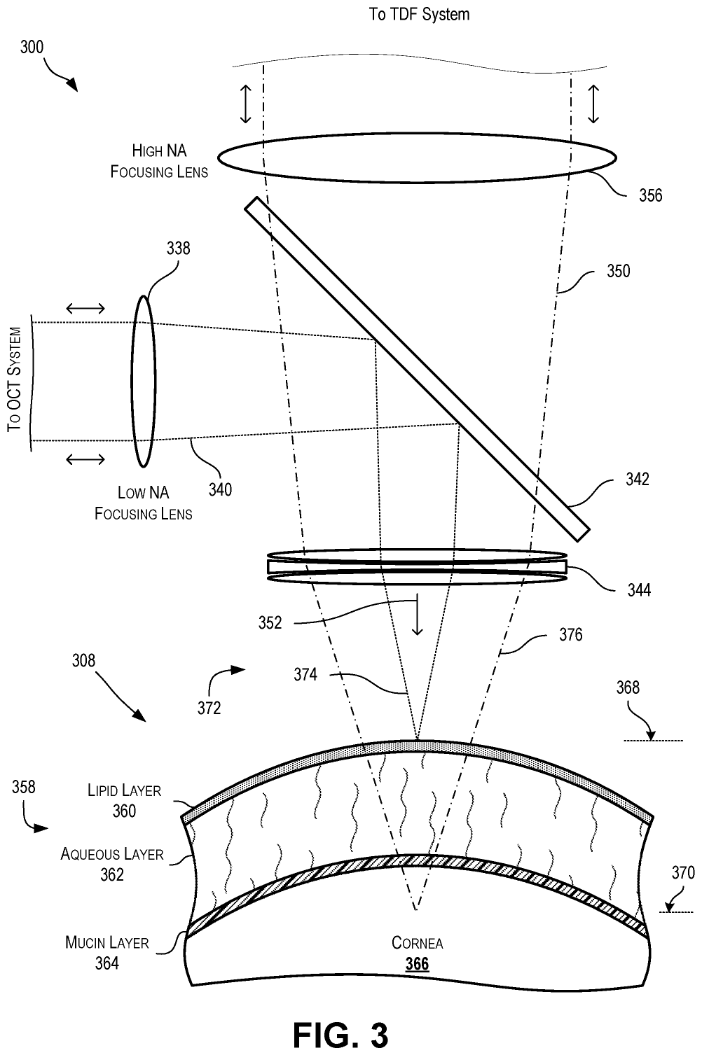

FIG. 3 is a schematic diagram depicting a dual focusing system 300 according to certain aspects of the present disclosure. The dual focusing system 300 can be the dual focusing system 206 of FIG. 2. The dual focusing system 300 can include an OCT focusing lens 338, a TDF focusing lens 356, a dichoric mirror 342, and an objective lens 344. The dual focusing system 300 can combine OCT sample light 340 and TDF sample light 350 into a combined beam 372. A first component 374 of the combined beam 372 can originate from the OCT sample light 340 and a second component 376 of the combined beam 372 can originate from the TDF sample light 350. The combined beam 372 can be directed onto an eye 308 to measure characteristics (e.g., thicknesses) of the tear film 358. The eye 308 can include a tear film 358 covering the cornea 366. The tear film 358 can include a mucin layer 364 at the surface of the cornea 366, a lipid layer 360 exposed to the surrounding environment (e.g., exposed to air), and an aqueous layer 362 between the mucin layer 364 and the lipid layer 360. The OCT sample light 340 can be used to measure the overall thickness of the tear film 358. The TDF sample light 340 can be used to measure the thickness of the lipid layer 360 of the tear film 358.

The dual focusing system 300 can accept OCT sample light 340 through an OCT focusing lens 338 from an optical coherence tomography system. The OCT focusing lens 338 can be a low numerical aperture lens. The OCT sample light 340 can reflect off a dichoric mirror 342 and through an objective lens 344 towards an eye 308. The OCT focusing lens 338 and objective lens 344 combined act to focus the OCT sample light 340 to an OCT focal plane 368 at a surface of a tear film 358, such as an outermost surface of the tear film 358 (e.g., the top of the lipid layer 360). The OCT focal plane 368 can be tangential to the surface of the lipid layer 360 and perpendicular to the measurement direction 352.

The dual focusing system 300 can accept TDF sample light 350 through an TDF focusing lens 356 from a thickness dependent fringe system. The TDF focusing lens 356 can be a high numerical aperture lens. The TDF sample light 350 can reflect off a dichoric mirror 342 and through the objective lens 344 towards the eye 308. The TDF focusing lens 356 and objective lens 344 combined act to focus the TDF sample light 350 to a TDF focal plane 370 beyond a surface of a tear film 358, such a distance within the eye 308. The TDF focal plane 370 can intersect the eye and be perpendicular to the measurement direction 352.

The distance between the OCT focal plane 368 and the TDF focal plane 370 can be any suitable distance, such as approximately 1 mm, 2 mm, or 3 mm. In some cases, the distance can be at or approximately 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, 11.5 mm, 12 mm, 12.5 mm, 13 mm, 13.5 mm, 14 mm, 14.5 mm, or 15 mm. In some cases, the distance can be between 0.1 mm and 15 mm, 0.5 mm and 3.5 mm, 1 mm and 3 mm, or 1 mm and 2 mm. Other distances may be used as appropriate. The distance can be selected to achieve desired performance of the thickness dependent fringe system.

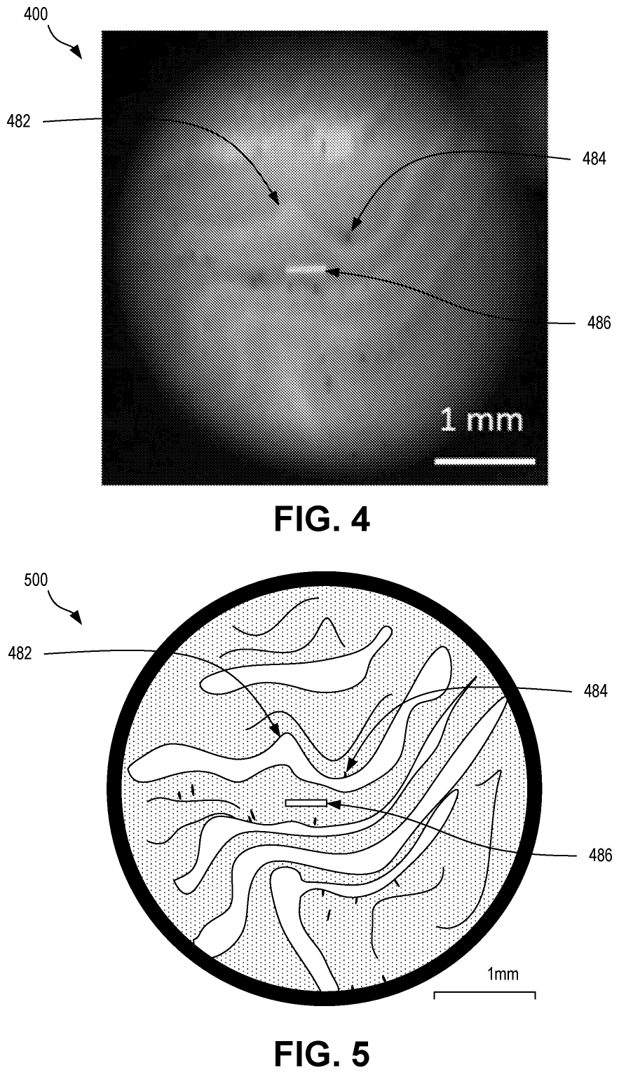

FIG. 4 is an image 400 of a tear film surface taken by a thickness dependent fringe system of a multimodal interferometric tear film measurement system according to certain aspects of the present disclosure. The image 400 can be taken using the multimodal interferometry system 100 or multimodal interferometry system 200 of FIG. 1 or 2. FIG. 5 is a graphic representation 500 of the image 400 of FIG. 4 according to certain aspects of the present disclosure.

Image 400 is an example of a single image captured using the system. In some cases, the system can capture individual images, several sequential images, or numerous sequential images (e.g., a video), such as at 30 frames per second. Video such as image 400 over time, such as while a patient is holding an eyelid open and/or blinking. When blinking, the features depicted in the image 400 can either move along with the surface of the tear film as the eyelid slides across the surface of the tear film, or can remain at the same location despite movement of the surface of the tear film during blinking.

The image 400 depicts various features, such as lines 482, spots 484, and a bright rectangle 486. The lines 482 can represent slight changes in thickness of the lipid layer of the tear film. The spots 484 can represent strong changes in thickness of the lipid layer of the tear film, such as due to debris or dry spots. Spots 484 that move between blinks are likely debris on or in the lipid layer, whereas spots 484 that remain stationary between blinks are likely dry spots on the lipid layer. The bright rectangle 486 is an artifact associated with the light from the optical coherence tomography system as it scans to produce measurements.

Characteristics of the normal and abnormal lipid layer cam be analyzed, including interference patterns, stability, and relation to the dynamics of the whole tear film. By comparing images of the lipid layer, such as image 400, with synchronous images of the entire tear film, further diagnostics can be performed.

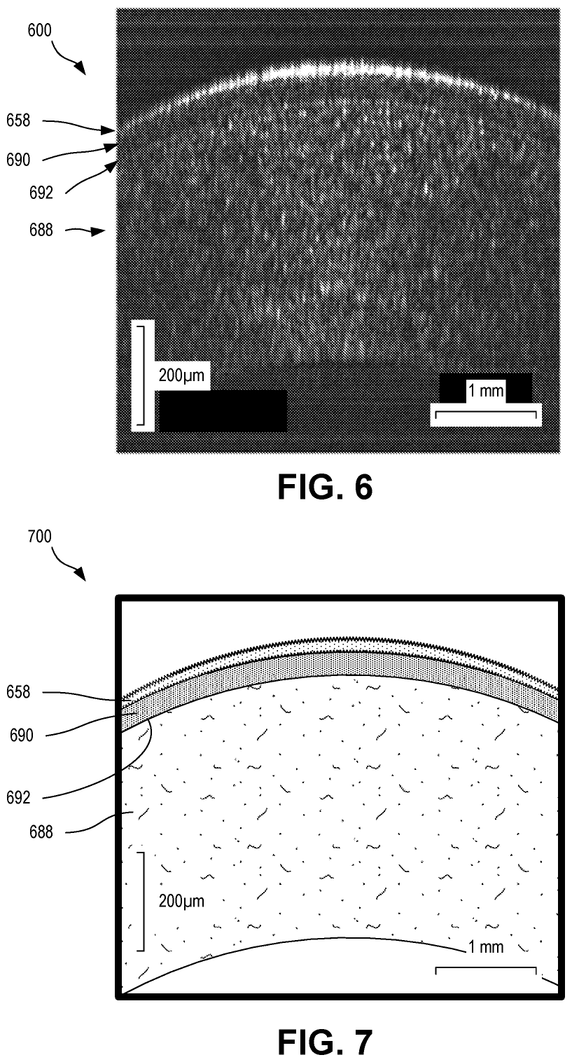

FIG. 6 is a cross-sectional image 600 of a tear film as taken by an optical coherence tomography system of a multimodal interferometric tear film measurement system according to certain aspects of the present disclosure. The image 600 can be taken using the multimodal interferometry system 100 or multimodal interferometry system 200 of FIG. 1 or 2. FIG. 7 is a graphic representation 700 of the image 600 of FIG. 6 according to certain aspects of the present disclosure.

Image 600 depicts various layers of an eye, including the tear film 658 and the cornea, including the stroma 688, the Bowman's membrane 692, and the epithelium 690. The image 600 can provide useful data for diagnosis and can be compared with the thickness dependent fringe measurements, such as those of image 400.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art.

As used below, any reference to a series of examples is to be understood as a reference to each of those examples disjunctively (e.g., “Examples 1-4” is to be understood as “Examples 1, 2, 3, or 4”).

Example 1 is a multimodal interferometric system, comprising: a first interferometric imaging system having a port for outputting a first light wave and receiving a first reflected light wave; a second interferometric imaging system having a port for outputting a second light wave and receiving a second reflected light wave, wherein the first light wave and the second light wave have different wavelengths; and a dual focusing system having: a first port optically coupled to the port of the first interferometric imaging system; a second port optically coupled to the port of the second interferometric imaging system; and a dichoric mirror optically coupled to the first port through a first focusing lens and the second port through a second focusing lens, wherein the dichoric mirror is selected to reflect the first light wave and the first reflected light wave while allowing light from the second light wave and the second reflected light wave to pass therethrough, wherein the dichoric mirror is positioned to combine the first light wave and the second light wave into a combined light wave and direct the combined light wave through an objective lens, wherein the first light wave passing through the first focusing lens and the objective lens is focused at a first focal plane, wherein the second light wave passing through the second focusing lens and the objective lens is focused at a second focal plane, and wherein the first focal plane and second focal plane are spaced apart from one another.

Example 2 is the system of example 1, wherein the first interferometric imaging system is an optical coherence tomography system including a low-coherence light source for generating the first light wave and a beam coupler for combining the first reflected light wave and a reference light wave.

Example 3 is the system of examples 1 or 2, wherein the second interferometric imaging system is a thickness dependent fringe system including a light source for generating the second light wave and a beam splitter for directing the second light wave from the light source to the port of the second interferometric imaging system, wherein the beam splitter is positioned to permit the second reflected light wave to pass therethrough and into a sensor.

Example 4 is the system of examples 1-3, wherein the first focal plane is positionable at a surface of a tear film of an eye, and wherein the second focal plane is positionable within the eye at a distance beyond the surface of the tear film of the eye.

Example 5 is the system of examples 1-4, wherein a distance between the first focal plane and the second focal plane is between one millimeter and fifteen millimeters.

Example 6 is the system of examples 1-5, wherein the first light wave has a first wavelength that is longer than a second wavelength of the second light wave, and wherein the dichoric mirror is a hot mirror that reflects light at the first wavelength and passes through light at the second wavelength.

Example 7 is the system of examples 1-6, further comprising a processor coupled to the first interferometric imaging system and the second interferometric imaging system to synchronously initiate interferometric measurements from both the first interferometric imaging system and the second interferometric imaging system.

Example 8 is a multimodal objective for simultaneous, multimodal interferometry, the multimodal objective comprising: a first port optically couplable to a first interferometric imaging system for receiving a first light wave and transmitting a first reflected light wave; a second port optically couplable to a second interferometric imaging system for receiving a second light wave and transmitting a second reflected light wave, wherein the first light wave and the second light wave have different wavelengths; and a dichoric mirror optically coupled to the first port through a first focusing lens and the second port through a second focusing lens, wherein the dichoric mirror is selected to reflect the first light wave and the first reflected light wave while allowing light from the second light wave and the second reflected light wave to pass therethrough, wherein the dichoric mirror is positioned to combine the first light wave and the second light wave into a combined light wave and direct the combined light wave through an objective lens, wherein the first light wave passing through the first focusing lens and the objective lens is focused at a first focal plane, wherein the second light wave passing through the second focusing lens and the objective lens is focused at a second focal plane, and wherein the first focal plane and second focal plane are spaced apart from one another.

Example 9 is the multimodal objective of example 8, wherein the first focal plane is positionable at a surface of a tear film of an eye, and wherein the second focal plane is positionable within the eye at a distance beyond the surface of the tear film of the eye.

Example 10 is the multimodal objective of examples 8 or 9, wherein a distance between the first focal plane and the second focal plane is between one millimeter and fifteen millimeters.

Example 11 is the multimodal objective of examples 8-10, wherein the first light wave has a first wavelength that is longer than a second wavelength of the second light wave, and wherein the dichoric mirror is a hot mirror that reflects light at the first wavelength and passes through light at the second wavelength.

Example 12 is the multimodal objective of examples 8-11, wherein the objective lens is an achromatic lens.

Example 13 is the multimodal objective of examples 8-12, wherein the first focusing lens and the second focusing lens are individually adjustable to respectively adjust the first focal plane and the second focal plane.

Example 14 is a method for performing simultaneous, multimodal interferometry, the method comprising: receiving a first light wave at a first port and a second light wave at a second port, wherein the first light wave is associated with a first interferometric technique and the second light wave is associated with a second interferometric technique; directing the first light wave to a dichoric mirror through a first focusing lens; directing the second light wave to the dichoric mirror through a second focusing lens; combining the first light wave and the second light wave using the dichoric mirror and directing the combined light to an objective lens, wherein the combined light includes a first component from the first light wave and a second component from the second light wave; outputting the combined light through the objective lens, wherein the first focusing lens and the objective lens focus the first component of the combined light onto a first focal plane, wherein the second focusing lens and the objective lens focus the second component of the combined light onto a second focal plane, and wherein the first focal plane and the second focal plane are spaced apart from one another; receiving combined reflected light at the objective lens and directing the combined reflected light to the dichoric mirror, wherein the combined reflected light is the combined light reflected off a subject; splitting the combined reflected light into a first reflected light wave and a second reflected light wave using the dichoric mirror, wherein the first reflected light is directed through the first focusing lens, and wherein the second reflected light is directed through the second focusing lens; and outputting the first reflected light at the first port and the second reflected light at the second port, wherein outputting of the first reflected light results in measurement data according to the first interferometric technique, and wherein outputting of the second reflected light results in measurement data according to the second interferometric technique.

Example 15 is the method of example 14, wherein receiving the first light wave at the first port and the second light wave at the second port occurs simultaneously.

Example 16 is the method of examples 14 or 15, further comprising: generating the first light wave at a low-coherence light source of an optical coherence tomography system; generating a reference light wave using the first light wave; combining the first reflected light wave and the reference light wave into a first combined measurement wave; and measuring interference patterns in the first combined measurement wave.

Example 17 is the method of examples 14-16, further comprising: generating the second light wave at a light source of a thickness dependent fringe interferometry system; reflecting the second light wave off a beam splitter and into the second port; and passing the second reflected light wave through the beam splitter and into a sensor.

Example 18 is the method of examples 14-17, further comprising maneuvering the first focusing lens, the second focusing lens, and the objective lens to position the first focal plane at a surface of a tear film of an eye and the second focal plane within the eye at a distance beyond the surface of the tear film of the eye.

Example 19 is the method of examples 14-18, wherein a distance between the first focal plane and the second focal plane is between one millimeter and fifteen millimeters.

Example 20 is the method of examples 14-19, further comprising: receiving a trigger signal, wherein receiving the trigger signal initiates a simultaneous measurement process and results in simultaneously receiving the first light wave at the first port and the second light wave at the second port; and simultaneously transmitting the measurement data associated with the first interferometric technique and the measurement data associated with the second interferometric technique.

What is claimed is:

1. A multimodal interferometric system, comprising:
a first interferometric imaging system having a port for outputting a first light wave and receiving a first reflected light wave;
a second interferometric imaging system having a port for outputting a second light wave and receiving a second reflected light wave, wherein the first light wave and the second light wave have different wavelengths; and
a dual focusing system having:
a first port optically coupled to the port of the first interferometric imaging system;
a second port optically coupled to the port of the second interferometric imaging system; and
a dichoric mirror optically coupled to the first port through a first focusing lens and the second port through a second focusing lens, wherein the dichoric mirror is selected to reflect the first light wave and the first reflected light wave while allowing light from the second light wave and the second reflected light wave to pass therethrough, wherein the dichoric mirror is positioned to combine the first light wave and the second light wave into a combined light wave and direct the combined light wave through an objective lens, wherein the first light wave passing through the first focusing lens and the objective lens is focused at a first focal plane, wherein the second light wave passing through the second focusing lens and the objective lens is focused at a second focal plane, and wherein the first focal plane and second focal plane are spaced apart from one another.

2. The system of claim 1, wherein the first interferometric imaging system is an optical coherence tomography system including a low-coherence light source for generating the first light wave and a beam coupler for combining the first reflected light wave and a reference light wave.

3. The system of claim 1, wherein the second interferometric imaging system is a thickness dependent fringe system including a light source for generating the second light wave and a beam splitter for directing the second light wave from the light source to the port of the second interferometric imaging system, wherein the beam splitter is positioned to permit the second reflected light wave to pass therethrough and into a sensor.

4. The system of claim 1, wherein the first focal plane is positionable at a surface of a tear film of an eye, and wherein the second focal plane is positionable within the eye at a distance beyond the surface of the tear film of the eye.

5. The system of claim 1, wherein a distance between the first focal plane and the second focal plane is between one millimeter and fifteen millimeters.

6. The system of claim 1, wherein the first light wave has a first wavelength that is longer than a second wavelength of the second light wave, and wherein the dichoric mirror is a hot mirror that reflects light at the first wavelength and passes through light at the second wavelength.

7. The system of claim 1, further comprising a processor coupled to the first interferometric imaging system and the second interferometric imaging system to synchronously initiate interferometric measurements from both the first interferometric imaging system and the second interferometric imaging system.

8. A multimodal objective for simultaneous, multimodal interferometry, the multimodal objective comprising:
a first port optically couplable to a first interferometric imaging system for receiving a first light wave and transmitting a first reflected light wave;
a second port optically couplable to a second interferometric imaging system for receiving a second light wave and transmitting a second reflected light wave, wherein the first light wave and the second light wave have different wavelengths; and
a dichoric mirror optically coupled to the first port through a first focusing lens and the second port through a second focusing lens, wherein the dichoric mirror is selected to reflect the first light wave and the first reflected light wave while allowing light from the second light wave and the second reflected light wave to pass therethrough, wherein the dichoric mirror is positioned to combine the first light wave and the second light wave into a combined light wave and direct the combined light wave through an objective lens, wherein the first light wave passing through the first focusing lens and the objective lens is focused at a first focal plane, wherein the second light wave passing through the second focusing lens and the objective lens is focused at a second focal plane, and wherein the first focal plane and second focal plane are spaced apart from one another.

9. The multimodal objective of claim 8, wherein the first focal plane is positionable at a surface of a tear film of an eye, and wherein the second focal plane is positionable within the eye at a distance beyond the surface of the tear film of the eye.

10. The multimodal objective of claim 8, wherein a distance between the first focal plane and the second focal plane is between one millimeter and fifteen millimeters.

11. The multimodal objective of claim 8, wherein the first light wave has a first wavelength that is longer than a second wavelength of the second light wave, and wherein the dichoric mirror is a hot mirror that reflects light at the first wavelength and passes through light at the second wavelength.

12. The multimodal objective of claim 8, wherein the objective lens is an achromatic lens.

13. The multimodal objective of claim 8, wherein the first focusing lens and the second focusing lens are individually adjustable to respectively adjust the first focal plane and the second focal plane.

14. A method for performing simultaneous, multimodal interferometry, the method comprising:
receiving a first light wave at a first port and a second light wave at a second port, wherein the first light wave is associated with a first interferometric technique and the second light wave is associated with a second interferometric technique;
directing the first light wave to a dichoric mirror through a first focusing lens;
directing the second light wave to the dichoric mirror through a second focusing lens;
combining the first light wave and the second light wave using the dichoric mirror and directing the combined light to an objective lens, wherein the combined light includes a first component from the first light wave and a second component from the second light wave;
outputting the combined light through the objective lens, wherein the first focusing lens and the objective lens focus the first component of the combined light onto a first focal plane, wherein the second focusing lens and the objective lens focus the second component of the combined light onto a second focal plane, and wherein the first focal plane and the second focal plane are spaced apart from one another;
receiving combined reflected light at the objective lens and directing the combined reflected light to the dichoric mirror, wherein the combined reflected light is the combined light reflected off a subject;
splitting the combined reflected light into a first reflected light wave and a second reflected light wave using the dichoric mirror, wherein the first reflected light is directed through the first focusing lens, and wherein the second reflected light is directed through the second focusing lens; and
outputting the first reflected light at the first port and the second reflected light at the second port, wherein outputting of the first reflected light results in measurement data according to the first interferometric technique, and wherein outputting of the second reflected light results in measurement data according to the second interferometric technique.

15. The method of claim 14, wherein receiving the first light wave at the first port and the second light wave at the second port occurs simultaneously.

16. The method of claim 14, further comprising:
generating the first light wave at a low-coherence light source of an optical coherence tomography system;
generating a reference light wave using the first light wave;
combining the first reflected light wave and the reference light wave into a first combined measurement wave; and
measuring interference patterns in the first combined measurement wave.

17. The method of claim 14, further comprising:
generating the second light wave at a light source of a thickness dependent fringe interferometry system;
reflecting the second light wave off a beam splitter and into the second port; and
passing the second reflected light wave through the beam splitter and into a sensor.

18. The method of claim 14, further comprising maneuvering the first focusing lens, the second focusing lens, and the objective lens to position the first focal plane at a surface of a tear film of an eye and the second focal plane within the eye at a distance beyond the surface of the tear film of the eye.

19. The method of claim 14, wherein a distance between the first focal plane and the second focal plane is between one millimeter and fifteen millimeters.

20. The method of claim 14, further comprising:
receiving a trigger signal, wherein receiving the trigger signal initiates a simultaneous measurement process and results in simultaneously receiving the first light wave at the first port and the second light wave at the second port; and
simultaneously transmitting the measurement data associated with the first interferometric technique and the measurement data associated with the second interferometric technique.
